(19) United States
(12) Patent Application Publication
(10) Pub. No.: US 2014/0213583 A1
(43) Pub. Date: Jul. 31, 2014

(54) PI3K Inhibitor for Use in the Treatment of Bone Cancer or for Preventing Metastatic Dissemination Primary Cancer Cells into the Bone

(71) Applicants: Carla De Giovanni, Bologna (IT); Pier-Luigi Lollini, Bologna (IT); Patrizia Nanni, Bologna (IT)

(72) Inventors: Carla De Giovanni, Bologna (IT); Pier-Luigi Lollini, Bologna (IT); Patrizia Nanni, Bologna (IT)

(73) Assignee: NOVARTIS AG, Basel (CH)

(21) Appl. No.: 14/240,505

(22) PCT Filed: Aug. 31, 2012
(86) PCT No.: PCT/EP2012/067019

§ 371 Date: Feb. 24, 2014

Related U.S. Application Data
(60) Provisional application No. 61/530,089, filed on Sep. 1, 2011.

(51) Int. Cl. C07D 401/04

(52) U.S. Cl. CPC C07D 401/04
USPC 514/232.2

(57) ABSTRACT

Provided herein are methods for the treatment of bone cancer and prevention of the metastatic spread of cancer.

### FIELD OF INVENTION

The present invention is related to methods for the treatment of bone cancer, as well as for the prevention of the metastatic spread of cancer.

### BACKGROUND

Metastatic dissemination is the most feared sequel of cancer, and the main cause of lethality. Cells leaving primary tumors can reach every organ and district in the body. However, each tumor type displays a specific metastatic pattern resulting from the interaction of tumor-intrinsic and organ-specific molecular and cellular properties (1, 2). For example, the most common targets of breast cancer dissemination are the bones, the lungs, abdominal viscera organs and the brain (3-6). Metastatic bone cancers result from metastatic dissemination from primary cancers, and are distinguished from hematological cancers, such as multiple myeloma and leukemia, which begins in the bone marrow. Metastatic patterns determine not only the duration of recurrence-free intervals, but more importantly, quality of life and, eventually, survival. The search for novel antimetastatic agents remains an important objective in oncology.

### SUMMARY OF THE INVENTION

Accordingly, in one aspect, provided herein is a method for the treatment of bone cancer in a subject in need of such treatment, comprising administering a compound according to formula (I), or a stereoisomer, tautomer, or pharmaceutically acceptable salt thereof, to the subject such that the bone cancer is treated:

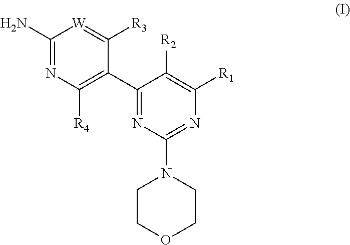

wherein W is CR_{w} or N,

wherein R_{w} is selected from the group consisting of:
(1) hydrogen,
(2) cyano,
(3) halogen,
(4) methyl,
5) trifluoromethyl,
(6) sulfonamide;

R₁ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(23) —SO₂NR₁ₐR_{1b},

wherein R₁ₐ, and R_{1b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;

R₂ is selected from the group consisting of:
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b},

wherein R₂ₐ, and R_{2b} are independently selected from the group consisting of:
(a) hydrogen, and
(b) substituted or unsubstituted alkyl;

R₃ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(14) —NR₃ₐR_{3b}
(13) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ,

wherein R₃ₐ, and R_{3b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and

R₄ is selected from the group consisting of
(1) hydrogen, and
(2) halogen.

In certain embodiments of the method, the bone cancer is selected from chondrosarcoma, osteosarcoma, Ewing's sarcoma, chordoma, fibrosarcoma, and malignant fibrous histiocytoma (MFH).

In another aspect, provided herein is a method of preventing the metastatic dissemination of primary cancer cells into the bone of a subject in need of such prevention, comprising administering a compound according to formula (I), or a stereoisomer, tautomer, or pharmaceutically acceptable salt thereof, to the subject such that the metastatic dissemination of primary cancer cells into the bone of the subject is prevented.

In one embodiment of the method of preventing, the primary cancer cells originate from cancers of the breast, lung, pancreas, kidney or prostate. In another embodiment, the primary cancer cells are breast cancer cells.

In another aspect, provided herein is a method for the treatment of bone cancer in a subject in need of such treatment, comprising administering a pharmaceutical composition comprising a compound according to formula (I), or a stereoisomer, tautomer, or pharmaceutically acceptable salt thereof, and a pharmaceutically acceptable carrier, diluent or excipient, to the subject such that the bone cancer is treated.

In yet another aspect, provided herein is a method of preventing the metastatic dissemination of primary cancer cells into the bone of a subject in need of such prevention, comprising administering a pharmaceutical composition comprising a compound according to formula (I), or a stereoisomer, tautomer, or pharmaceutically acceptable salt thereof, and a pharmaceutically acceptable carrier, diluent or excipient, to the subject such that the metastatic dissemination of primary cancer cells into the bone of the subject is prevented.

In certain embodiments of the foregoing methods of treatment and methods of preventing,
W represents CH;
R¹ represents N-morpholinyl;
R² represents hydrogen;
R³ represents trifluoromethyl; and
R⁴ represents hydrogen.

In still another aspect, provided herein is a method for the treatment of bone cancer in a subject in need of such treatment, comprising administering Compound A, or a stereoisomer, tautomer, or pharmaceutically acceptable salt thereof, to the subject such that the bone cancer is treated:

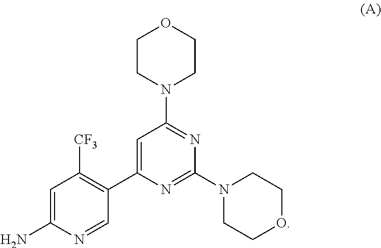 [SMILES: Nc1cc(C(F)(F)F)c(-c2cc(N3CCOCC3)nc(N3CCOCC3)n2)cn1]

In certain embodiments of the foregoing methods of treatment of bone cancer, the bone cancer is metastatic bone cancer.

In a particular aspect, provided herein is a method of preventing the metastatic dissemination of primary cancer cells into the bone of a subject in need of such prevention, comprising administering Compound A, or a stereoisomer, tautomer, or pharmaceutically acceptable salt thereof, to the subject such that the metastatic dissemination of primary cancer cells into bone of the subject is prevented.

### BRIEF DESCRIPTION OF THE DRAWINGS

FIG. 1 depicts multiorgan inhibition of 453-EGFP metastatic growth by Compound A. (A) Incidence of metastases in different sites; a significant inhibition by Compound A was recorded in the brain, bone marrow and liver, p<0.05 at least, Fisher's exact test. Representative samples of dissected control and treated mouse brains (ventral view, B) and lungs (C) showing reduction in metastatic burden by Compound A.

FIG. 2 depicts quantitative analysis of antimetastatic activity of Compound A. Each bar represents the mean and SEM of groups of 6-9 mice treated i.v. with 453-EGFP cells, percentage inhibition is shown in each graph above Compound A bar. (A) Metastatic burden in the brain as evaluated by qPCR, see Materials and Methods for calculations; (B, D) Cytofluorometric determination of HER-2-positive metastatic cells in the dissociated brain (B) and femural bone marrow (D); (C, E, F) Visual count of metastatic sites per mouse. Statistical evaluation of metastasis inhibition by Compound A: panels A, B, C, F, p<0.05 at least by the Student's t test.

### DETAILED DESCRIPTION

### I. Methods of Treatment

Common targets of metastatic dissemination include the bones, the lungs, the brain, and abdominal viscera organs (e.g., the stomach, liver, intestines, spleen, pancreas, and parts of the urinary and reproductive tracts).

Metastasis and metastatic dissemination, according to the present disclosure, are understood to mean the spread of cancer cells from an original site to another part of the body. The formation of metastasis is a very complex process and depends on detachment of malignant cells from the primary tumor, invasion of the extracellular matrix, penetration of the endothelial basement membranes to enter the body cavity and vessels, and then, after being transported by the blood, infiltration of target organs. Finally, the growth of a new tumor at the target site depends on angiogenesis. Tumor metastasis often occurs even after the removal of the primary tumor because tumor cells or components may remain and develop metastatic potential. In one embodiment, the term “metastasis” according to the invention relates to “distant metastasis” which relates to a metastasis which is remote from the primary tumor.

The present disclosure also relates to methods of treating and preventing bone cancer, a condition characterized by an abnormal growth of malignant cells on or within the bone, excluding cancers originating in the bone marrow (e.g., hematological cancers such as multiple myeloma and leukemia). Non-limiting examples of bone cancer include chondrosarcoma, osteosarcoma, Ewing's sarcoma, chordoma, fibrosarcoma, and malignant fibrous histiocytoma (MFH). Cancers that originate on or within the bone are referred to as primary bone cancers. Cancers of the bone that originate in another part of the body (such as the breast, lungs, or colon) are secondary, or metastatic, bone cancers.

Thus, in one aspect, provided herein is a method for the treatment of bone cancer in a subject in need of such treatment, comprising administering a compound according to formula (I), or a stereoisomer, tautomer, or pharmaceutically acceptable salt thereof, to the subject such that the bone cancer is treated:

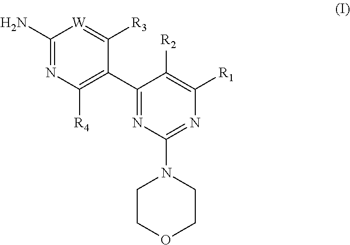

wherein W is CR_{w} or N,

wherein R_{w} is selected from the group consisting of: (1) hydrogen, (2) cyano, (3) halogen, (4) methyl, (5) trifluoromethyl, (6) sulfonamide;

R₁ is selected from the group consisting of: (1) hydrogen, (2) cyano, (3) nitro, (4) halogen, (5) substituted and unsubstituted alkyl, (6) substituted and unsubstituted alkenyl, (7) substituted and unsubstituted alkynyl, (8) substituted and unsubstituted aryl, (9) substituted and unsubstituted heteroaryl, (10) substituted and unsubstituted heterocyclyl, (11) substituted and unsubstituted cycloalkyl, (12) —COR_{N}, (13) —CO₂R₁ₐ, (14) —CONR₁ₐR_{1b}, (15) —NR₁ₐR_{1b}, (16) —NR₁ₐCOR_{1b}, (17) —NR₁ₐSO₂R_{1b}, (18) —OCOR₁ₐ, (19) —OR₁ₐ, (20) —SR₁ₐ, (21) —SOR₁ₐ, (23) —SO₂NR₁ₐR_{1b}, wherein R₁ₐ, and R_{1b} are independently selected from the group consisting of: (a) hydrogen, (b) substituted or unsubstituted alkyl, (c) substituted and unsubstituted aryl, (d) substituted and unsubstituted heteroaryl, (e) substituted and unsubstituted heterocyclyl, and (f) substituted and unsubstituted cycloalkyl;

R₂ is selected from the group consisting of: (1) hydrogen, (2) cyano, (3) nitro, (4) halogen, (5) hydroxy, (6) amino, (7) substituted and unsubstituted alkyl, (8) —COR₂ₐ, and (9) —NR₂ₐCOR_{2b}, wherein R₂ₐ, and R_{2b} are independently selected from the group consisting of: (a) hydrogen, and (b) substituted or unsubstituted alkyl;

R₃ is selected from the group consisting of: (1) hydrogen, (2) cyano, (3) nitro, (4) halogen, (5) substituted and unsubstituted alkyl, (6) substituted and unsubstituted alkenyl, (7) substituted and unsubstituted alkynyl, (8) substituted and unsubstituted aryl, (9) substituted and unsubstituted heteroaryl, (10) substituted and unsubstituted heterocyclyl, (11) substituted and unsubstituted cycloalkyl, (12) —COR₃ₐ, (14) —NR₃ₐR_{3b}, (13) —NR₃ₐCOR_{3b}, (15) —NR₃ₐSO₂R_{3b}, (16) —OR₃ₐ, (17) —SR₃ₐ, (18) —SOR₃ₐ, (19) —SO₂R₃ₐ, wherein R₁ₐ, and R_{3b} are independently selected from the group consisting of: (a) hydrogen, (b) substituted or unsubstituted alkyl, (c) substituted and unsubstituted aryl, (d) substituted and unsubstituted heteroaryl, (e) substituted and unsubstituted heterocyclyl, and (f) substituted and unsubstituted cycloalkyl; and

R₄ is selected from the group consisting of (1) hydrogen, and (2) halogen.

In one embodiment of the method for the treatment of bone cancer, the bone cancer is metastatic bone cancer.

In another embodiment of the method for the treatment of bone cancer, W represents CH, R¹ represents substituted and unsubstituted heterocyclyl, R² represents hydrogen, R³ represents substituted and unsubstituted alkyl, and R⁴ represents hydrogen. In another embodiment, W represents CH, R¹ represents N-morpholinyl, R² represents hydrogen, R³ represents trifluoromethyl, and R⁴ represents hydrogen.

In certain embodiments of the method, the bone cancer is selected from chondrosarcoma, osteosarcoma, Ewing's sarcoma, chordoma, fibrosarcoma, and malignant fibrous histiocytoma (MFH).

In another aspect, provided herein is a method of preventing the metastatic dissemination of primary cancer cells in a subject in need of such prevention, comprising administering a compound according to formula (I), or a stereoisomer, tautomer, or pharmaceutically acceptable salt thereof, to the subject such that the metastatic dissemination of cancer cells is prevented.

In one embodiment of the method of preventing the metastatic dissemination of primary cancer cells, the primary cancer cells originate from cancers of the breast, lung, pancreas, kidney or prostate. In another embodiment, the primary cancer cells are breast cancer cells. In still another embodiment, the primary cancer cells are prevented from disseminating into organs selected from bone, lungs, abdominal viscera organs, and the brain. In a particular embodiment, the primary cancer cells are prevented from disseminating into the bone of the subject.

Thus, in another aspect, provided herein is a method of preventing the metastatic dissemination of primary cancer cells into the bone of a subject in need of such prevention, comprising administering a compound according to formula (I), or a stereoisomer, tautomer, or pharmaceutically acceptable salt thereof, to the subject such that the metastatic dissemination of primary cancer cells into the bone of the subject is prevented.

In certain embodiments of the methods of preventing the metastatic dissemination of primary cancer cells, W represents CH, R¹ represents substituted and unsubstituted heterocyclyl, R² represents hydrogen, R³ represents substituted and unsubstituted alkyl, and R⁴ represents hydrogen. In other embodiments, W represents CH, R¹ represents N-morpholinyl, R² represents hydrogen, R³ represents trifluoromethyl, and R⁴ represents hydrogen.

In another aspect, provided herein is a method for the treatment of bone cancer in a subject in need of such treatment, comprising administering Compound A, or a stereoisomer, tautomer, or pharmaceutically acceptable salt thereof, to the subject such that the bone cancer is treated:

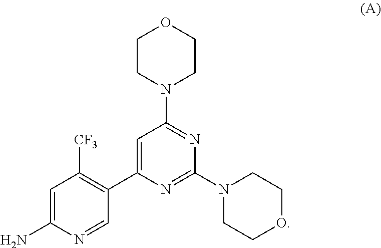 [SMILES: Nc1cc(C(F)(F)F)c(-c2cc(N3CCOCC3)nc(N3CCOCC3)n2)cn1]

In one embodiment of the method for the treatment of bone cancer, the bone cancer is metastatic bone cancer.

In a particular aspect, provided herein is a method of preventing the metastatic dissemination of primary cancer cells into the bone of a subject in need of such prevention, comprising administering Compound A, or a stereoisomer, tautomer, or pharmaceutically acceptable salt thereof, to the subject such that the metastatic dissemination of primary cancer cells into bone of the subject is prevented

In a preferred embodiment, the compound of formula (I) is the pan-phosphatidylinositol 3-kinase (PI3K) inhibitor 5-(2,6-di-morpholin-4-yl-pyrimidin-4-yl)-4-trifluoromethyl-pyridin-2-ylamine (referred to herein as “Compound A”).

The synthesis of Compound A is described in WO 2007/084786 (see Example 10, pages 139-140), the contents of which are incorporated herein by reference. Unless otherwise specified, or clearly indicated by the text, reference to compound of formula I of the invention includes both the free base of the compound, and all pharmaceutically acceptable salts of the compound.

### II. Pharmaceutical Compositions

In another aspect, provided herein is a method for the treatment of bone cancer in a subject in need of such treatment, comprising administering a pharmaceutical composition comprising a compound according to formula (I), or a stereoisomer, tautomer, or pharmaceutically acceptable salt thereof, and a pharmaceutically acceptable carrier, diluent or excipient, to the subject such that the bone cancer is treated.

In one embodiment of the method for the treatment of bone cancer, the bone cancer is metastatic bone cancer.

In yet another aspect, provided herein is a method of preventing the metastatic dissemination of primary cancer cells into the bone of a subject in need of such prevention, comprising administering a pharmaceutical composition comprising a compound according to formula (I), or a stereoisomer, tautomer, or pharmaceutically acceptable salt thereof, and a pharmaceutically acceptable carrier, diluent or excipient, to the subject such that the metastatic dissemination of primary cancer cells into the bone of the subject is prevented.

The compounds of the invention are useful in vitro or in vivo in treating metastatic dissemination. The compounds may be used alone or in compositions together with a pharmaceutically acceptable carrier or excipient. Pharmaceutical compositions of the present invention comprise a therapeutically effective amount of a phosphatidylinositol 3-kinase inhibitor compound described herein formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Other suitable pharmaceutically acceptable excipients are described in “Remington's Pharmaceutical Sciences,” Mack Pub. Co., New Jersey, 1991, incorporated herein by reference.

The compounds of the present invention may be administered to humans and other animals orally, parenterally, sublingually, by aerosolization or inhalation spray, rectally, intracisternally, intravaginally, intraperitoneally, bucally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Methods of formulation are well known in the art and are disclosed, for example, in Remington: The Science and Practice of Pharmacy, Mack Publishing Company, Easton, Pa., 19th Edition (1995). Pharmaceutical compositions for use in the present invention can be in the form of sterile, non-pyrogenic liquid solutions or suspensions, coated capsules, suppositories, lyophilized powders, transdermal patches or other forms known in the art.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-propanediol or 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form.

Alternatively, delayed absorption of a parenterally administered drug form may be accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also be prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, EtOAc, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Effective amounts of the compounds of the invention generally include any amount sufficient to detectably treat or prevent bone cancer or metastatic dissemination of cancer cells by any of the assays described herein, by other assays known to those having ordinary skill in the art, or by detecting an inhibition or alleviation of symptoms of bone cancer or metastatic dissemination of cancer cells. The amount of active ingredient that may be combined with the carrier materials to produce a single dosage form will vary depending upon the host treated and the particular mode of administration.

It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

According to the methods of treatment of the present invention, metastatic dissemination is reduced or prevented in a patient such as a human or lower mammal by administering to the patient a therapeutically effective amount of a compound of the invention, in such amounts and for such time as is necessary to achieve the desired result.

### III. Definitions

“Treating” is used herein to mean the reduction or alleviation of symptoms associated with a disorder or disease, or halt of further progression or worsening of those symptoms, or prevention or prophylaxis of the disease or disorder. For example, within the context of treating patients in need of treatment of bone cancer, successful treatment may include prevention of the occurrence of bone cancer, or a prevention of the metastatic dissemination of cancerous cells into bone, an alleviation of symptoms related to a cancerous growth or tumor in the bone, proliferation of capillaries, or diseased tissue, a halting in capillary proliferation, or a halting in the progression of a disease such as cancer or in the growth of cancerous cells. Treatment may also include administering the compounds used in the present invention in combination with other therapies. For example, the compounds and pharmaceutical formulations of the present invention may be administered before, during, or after surgical procedure and/or radiation therapy. The compounds of the invention can also be administered in conjunction with other anti-cancer drugs including those used in antisense and gene therapy.

As used herein, “limit”, “treat” and “treatment” are interchangeable terms as are “limiting” and “treating” and, as used herein, include preventative (e.g., prophylactic) and palliative treatment or the act of providing preventative or palliative treatment.

The term “subject” is intended to include animals. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from metastatic bone cancer.

By a “therapeutically effective amount” of a compound of the invention is meant a sufficient amount of the compound to treat or prevent metastatic dissemination, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

For purposes of the present invention, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose. In general, treatment regimens according to the present invention comprise administration to a patient in need of such treatment from about 10 mg to about 2000 mg of the compound(s) of this invention per day in single or multiple doses.

The term “dose range” as used herein refers to an upper and a lower limit of an acceptable variation of the amount of agent specified. Typically, a dose of the agent in any amount within the specified range can be administered to patients undergoing treatment.

The term “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

As used herein, the term “alkyl” refers to alkyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)—CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂—CH(CH₃)₂, —CH(CH₃)CH(CH₃)CH(CH₃)₂, —CH(CH₂CH₃)CH(CH₃)CH(CH₃)(CH₂CH₃), and others. Thus the phrase “alkyl groups” includes primary alkyl groups, secondary alkyl groups, and tertiary alkyl groups. Preferred alkyl groups include straight and branched chain alkyl groups having 1 to 12 carbon atoms or 1 to 6 carbon atoms.

The term “alkenyl” refers to straight chain, branched, or cyclic groups from 2 to about 20 carbon atoms such as those described with respect to alkyl groups as defined above, except having one or more carbon-carbon double bonds. Examples include, but are not limited to vinyl, —CH═C(H)(CH₃), —CH═C(CH₃)₂, —C(CH₃)═C(H)₂, —C(CH₃)═C(H)(CH₃), —C(CH₂CH₃)═CH₂, cyclohexenyl, cyclopentenyl, cyclohexadienyl, butadienyl, pentadienyl, and hexadienyl among others. Preferred alkenyl groups include straight chain and branched alkenyl groups and cyclic alkenyl groups having 2 to 12 carbon atoms or 2 to 6 carbon atoms.

The term “alkynyl” refers to straight chain, branched, or cyclic groups from 2 to about 20 carbon atoms such as those described with respect to alkyl groups as defined above, except having one or more carbon-carbon triple bonds. Examples include, but are not limited to —C≡C(H), —C≡C(CH₃), —C≡C(CH₂CH₃), —C(H₂)C≡C(H), —C(H)₂C≡C(CH₃), and —C(H)₂C≡C(CH₂CH₃) among others. Preferred alkynyl groups include straight chain and branched alkynyl groups having 2 to 12 carbon atoms or 2 to 6 carbon atoms.

Alkyl, alkenyl, and alkynyl groups may be substituted. “Substituted alkyl” refers to an alkyl group as defined above in which one or more bonds to a carbon(s) or hydrogen(s) are replaced by a bond to non-hydrogen and non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a higher-order bond (e.g., a double- or triple-bond) to a heteroatom such as oxygen in oxo, carbonyl, carboxyl, and ester groups; nitrogen in groups such as imines, oximes, hydrazones, and nitriles. Substituted alkyl groups further include alkyl groups in which one or more bonds to a carbon(s) or hydrogen(s) atoms is replaced by a bond to an aryl, heteroaryl, heterocyclyl, or cycloalkyl group. Preferred substituted alkyl groups include, among others, alkyl groups in which one or more bonds to a carbon or hydrogen atom is/are replaced by one or more bonds to fluoro, chloro, or bromo group. Another preferred substituted alkyl group is the trifluoromethyl group and other alkyl groups that contain the trifluoromethyl group. Other preferred substituted alkyl groups include those in which one or more bonds to a carbon or hydrogen atom is replaced by a bond to an oxygen atom such that the substituted alkyl group contains a hydroxyl, alkoxy, or aryloxy group. Other preferred substituted alkyl groups include alkyl groups that have an amine, or a substituted or unsubstituted alkylamine, dialkylamine, arylamine, (alkyl)(aryl)amine, diarylamine, heterocyclylamine, diheterocyclylamine, (alkyl)(heterocyclyl)amine, or (aryl)(heterocyclyl)amine group. Still other preferred substituted alkyl groups include those in which one or more bonds to a carbon(s) or hydrogen(s) atoms is replaced by a bond to an aryl, heteroaryl, heterocyclyl, or cycloalkyl group. Examples of substituted alkyl are: —(CH₂)₃NH₂, —(CH₂)₃NH(CH₃), —(CH₂)₃NH(CH₃)₂, —CH₂C(—CH₂)CH₂NH₂, —CH₂C(═O)CH₂NH₂, —CH₂S(═O)₂CH₃, —CH₂OCH₂NH₂, —CO₂H. Examples of substituents of substituted alkyl are: —CH₃, —C₂H₅, —CH₂OH, —OH, —OCH₃, —OC₂H₅, —OCF₃, —OC(═O)CH₃, —OC(═O)NH₂, —OC(═O)N(CH₃)₂, —CN, —NO₂, —C(═O)CH₃, —CO₂H, —CO₂CH₃, —CONH₂, —NH₂, —N(CH₃)₂, —NHSO₂CH₃, —NHCOCH₃, —NHC(═O)OCH₃, —NHSO—₂CH₃, —SO₂CH₃, —SO₂NH₂, halo.

The term “substituted alkenyl” has the same meaning with respect to alkenyl groups that substituted alkyl groups had with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

The term “substituted alkynyl” has the same meaning with respect to alkynyl groups that substituted alkyl groups had with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

The term “alkoxy” refers to RO— wherein R is alkyl. Representative examples of alkoxy groups include methoxy, ethoxy, t-butoxy, trifluoromethoxy, and the like.

The term “halogen” or “halo” refers to chloro, bromo, fluoro, and iodo groups. The term “haloalkyl” refers to an alkyl radical substituted with one or more halogen atoms. The term “haloalkoxy” refers to an alkoxy radical substituted with one or more halogen atoms.

The term “alkoxyalkyl” refers to the group -alk₁-O-alk₂ where alk₁ is alkyl or alkenyl, and alk₂ is alkyl or alkenyl. The term “aryloxyalkyl” refers to the group -alkyl O-aryl. The term “aralkoxyalkyl” refers to the group -alkylenyl-O-aralkyl.

The term “carbonyl” refers to the divalent group —C(O)—.

The term “cycloalkyl” refers to a mono- or polycyclic, heterocyclic or carbocyclic alkyl substituent. Representative cycloalkyl groups include cyclopropyl, cyclobutyl, cyclopentyl, cyclohexyl, cycloheptyl, and cyclooctyl and such rings substituted with straight and branched chain alkyl groups as defined above. Typical cycloalkyl substituents have from 3 to 8 backbone (i.e., ring) atoms in which each backbone atom is either carbon or a heteroatom. The term “heterocycloalkyl” refers herein to cycloalkyl substituents that have from 1 to 5, and more typically from 1 to 4 heteroatoms in the ring structure. Suitable heteroatoms employed in compounds of the present invention are nitrogen, oxygen, and sulfur. Representative heterocycloalkyl moieties include, for example, morpholino, piperazinyl, piperadinyl, and the like. Carbocycloalkyl groups are cycloalkyl groups in which all ring atoms are carbon. When used in connection with cycloalkyl substituents, the term “polycyclic” refers herein to fused and non-fused alkyl cyclic structures.

The term “aryl” refers to optionally substituted monocyclic and polycyclic aromatic groups having from 3 to 14 backbone carbon or hetero atoms, and includes both carbocyclic aryl groups and heterocyclic aryl groups. The term refers to, but is not limited to, groups such as phenyl, biphenyl, anthracenyl, naphthenyl by way of example. Carbocyclic aryl groups are aryl groups in which all ring atoms in the aromatic ring are carbon. The term “heteroaryl” refers herein to aryl groups having from 1 to 4 heteroatoms as ring atoms in an aromatic ring with the remainder of the ring atoms being carbon atoms.

The term “unsubstituted aryl” includes groups containing condensed rings such as naphthalene. It does not include aryl groups that have other groups such as alkyl or halo groups bonded to one of the ring members, as aryl groups such as tolyl are considered herein to be substituted aryl groups as described below. A preferred unsubstituted aryl group is phenyl. Unsubstituted aryl groups may be bonded to one or more carbon atom(s), oxygen atom(s), nitrogen atom(s), and/or sulfur atom(s) in the parent compound, however.

The term “substituted aryl group” has the same meaning with respect to unsubstituted aryl groups that substituted alkyl groups had with respect to unsubstituted alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g., dihydronaphthyl or tetrahydronaphthyl). Thus, the phrase “substituted aryl” includes, but is not limited to tolyl, and hydroxyphenyl among others.

The term “substituted heteroaryl” as used herein refers to a heteroaryl group as defined herein substituted by independent replacement of one, two or three of the hydrogen atoms thereon with Cl, Br, F, I, —OH, —CN, C₁-C₃-alkyl, C₁-C₆-alkoxy, C₁-C₆-alkoxy substituted with aryl, haloalkyl, thioalkoxy, amino, alkylamino, dialkylamino, mercapto, nitro, carboxaldehyde, carboxy, alkoxycarbonyl and carboxamide. In addition, any one substituent may be an aryl, heteroaryl, or heterocycloalkyl group.

The term “substituted heterocycle,” “heterocyclic group,” “heterocycle,” or “heterocyclyl,” as used herein refers to any 3- or 4-membered ring containing a heteroatom selected from nitrogen, oxygen, and sulfur or a 5- or 6-membered ring containing from one to three heteroatoms selected from the group consisting of nitrogen, oxygen, or sulfur; wherein the 5-membered ring has 0-2 double bonds and the 6-membered ring has 0-3 double bonds; wherein the nitrogen and sulfur atom maybe optionally oxidized; wherein the nitrogen and sulfur heteroatoms maybe optionally quarternized; and including any bicyclic group in which any of the above heterocyclic rings is fused to a benzene ring or another 5- or 6-membered heterocyclic ring independently defined above. Examples of heterocyclyl groups include, but are not limited to: unsaturated 3- to 8-membered rings containing 1 to 4 nitrogen atoms such as, but not limited to pyrrolyl, dihydropyridyl, pyrimidyl, pyrazinyl, tetrazolyl, (e.g., 1H-tetrazolyl, 2H-tetrazolyl); condensed unsaturated heterocyclic groups containing 1 to 4 nitrogen atoms such as, but not limited to, isoindolyl, indolinyl, indolizinyl, quinolyl, indazolyl; unsaturated 3- to 8-membered rings containing 1 to 2 oxygen atoms and 1 to 3 nitrogen atoms such as, but not limited to, oxadiazolyl (e.g., 1,2,4-oxadiazolyl, 1,3,4-oxadiazolyl, 1,2,5-oxadiazolyl); saturated 3- to 8-membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxadiazolyl, benzoxazinyl (e.g., 2H-1,4-benzoxazinyl); unsaturated 3- to 8-membered rings containing 1 to 3 sulfur atoms and 1 to 3 nitrogen atoms such as, but not limited to, thiadiazolyl (e.g., 1,2,3-thiadiazolyl, 1,2,4-thiadiazolyl, 1,3,4-thiadiazolyl, 1,2,-thiadiazolyl); saturated 3- to 8-membered rings containing 1 to 2 sulfur atoms and 1 to 3 nitrogen atoms such as, but not limited to, thiazolodinyl; saturated and unsaturated 3- to 8-membered rings containing 1 to 2 sulfur atoms such as, but not limited to, dihydrodithienyl, dihydrodithionyl, tetrahydrothiophene, tetra-hydrothiopyran; unsaturated condensed heterocyclic rings containing 1 to 2 sulfur atoms and 1 to 3 nitrogen atoms such as, but not limited to, benzothiadiazolyl, benzothiazinyl (e.g., 2H-1,4-benzothiazinyl), dihydrobenzothiazinyl (e.g., 2H-3,4-dihydrobenzothiazinyl), unsaturated 3- to 8-membered rings containing oxygen atoms such as, but not limited to furyl; unsaturated condensed heterocyclic rings containing 1 to 2 oxygen atoms such as benzodioxoyl (e.g., 1,3-benzodioxoyl); unsaturated 3- to 8-membered rings containing an oxygen atom and 1 to 2 sulfur atoms such as, but not limited to, dihydrooxathienyl; saturated 3- to 8-membered rings containing 1 to 2 oxygen atoms and 1 to 2 sulfur atoms such as 1,4-oxathiane; unsaturated condensed rings containing 1 to 2 sulfur atoms such as benzodithienyl; and unsaturated condensed heterocyclic rings containing an oxygen atom and 1 to 2 oxygen atoms such as benzoxathienyl. Preferred heterocycles include, for example: diazapinyl, pyrryl, pyrrolinyl, pyrrolidinyl, pyrazolyl, pyrazolinyl, pyrazolidinyl, imidazoyl, imidazolinyl, imidazolidinyl, pyridyl, piperidinyl, pyrazinyl, piperazinyl, N-methyl piperazinyl, azetidinyl, N-methylazetidinyl, pyrimidinyl, pyridazinyl, oxazolyl, oxazolidinyl, isoxazolyl, isoxazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, triazolyl, and benzothienyl. Heterocyclyl groups also include those described above in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1-dioxide. Preferred heterocyclyl groups contain 5 or 6 ring members. More preferred heterocyclyl groups include piperazine, 1,2,3-triazole, 1,2,4-triazole, tetrazole, thiomorpholine, homopiperazine, oxazolidin-2-one, pyrrolidin-2-one, quinuclidine, and tetrahydrofuran.

Heterocyclic moieties can be unsubstituted or monosubstituted or disubstituted with various substituents independently selected from hydroxy, halo, oxo (C=0), alkylimino wherein R is alkyl or alkoxy group), amino, alkylamino, dialkylamino, acylaminoalkyl, alkoxy, thioalkoxy, polyalkoxy, alkyl, cycloalkyl or haloalkyl. “Unsubstituted heterocyclyl” includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups.

The heterocyclic groups may be attached at various positions as will be apparent to those having skill in the organic and medicinal chemistry arts in conjunction with the disclosure herein. Non-limiting examples of heterocyclic groups also include the following:

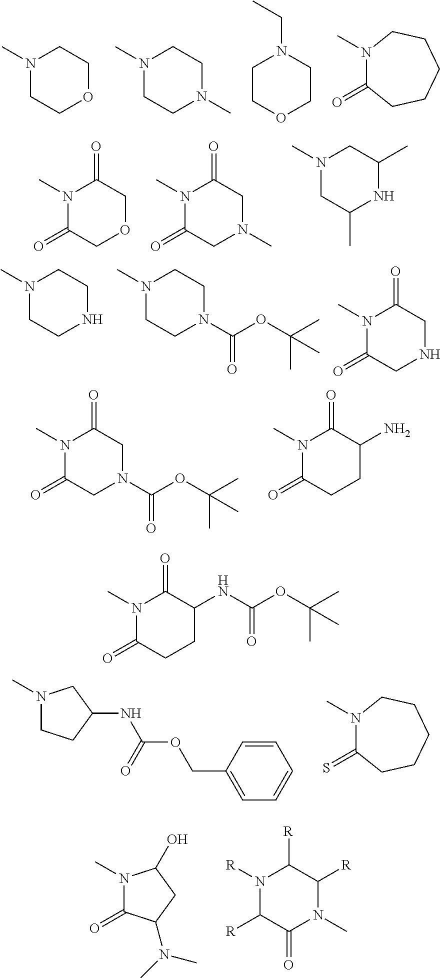

where R is H or a heterocyclic substituent, as described herein.

Representative heterocyclics include, for example, imidazolyl, pyridyl, piperazinyl, azetidinyl, thiazolyl, furanyl, triazolyl benzimidazolyl, benzothiazolyl, benzoxazolyl, quinolinyl, isoquinolinyl, quinazolinyl, quinoxalinyl, phthalazinyl, indolyl, to naphthpyridinyl, indazolyl, and quinolizinyl.

The term “optionally substituted” or “substituted” refers to the replacement of hydrogen with one or more monovalent or divalent radical. Suitable substitution groups include, for example, hydroxyl, nitro, amino, imino, cyano, halo, thio, sulfonyl, thioamido, amidino, imidino, oxo, oxamidino, methoxamidino, imidino, guanidino, sulfonamido, carboxyl, formyl, alkyl, substituted alkyl, haloalkyl, alkyamino, haloalkylamino, alkoxy, haloalkoxy, alkoxy-alkyl, alkylcarbonyl, aminocarbonyl, arylcarbonyl, aralkylcarbonyl, heteroarylcarbonyl, heteroaralkyl-carbonyl, alkylthio, aminoalkyl, cyanoalkyl, aryl, benzyl, pyridyl, pyrazolyl, pyrrole, thiophene, imidazolyl, and the like.

The substitution group can itself be substituted. The group substituted onto the substitution group can be carboxyl, halo, nitro, amino, cyano, hydroxyl, alkyl, alkoxy, aminocarbonyl, —SR, thioamido, —SO₃H, —SO₂R, or cycloalkyl, where R is typically hydrogen, hydroxyl or alkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms.

Representative substituted aminocarbonyl groups include, for example, those shown below. These can be further substituted by heterocyclyl groups and heteroaryl groups as will be apparent to those having skill in the organic and medicinal chemistry arts in conjunction with the disclosure herein. Preferred aminocarbonyl groups include: N-(2-cyanoethyl)carboxamide, N-(3-methoxypropyl)carboxamide, N-cyclopropylcarboxamide, N-(2-hydroxy-isopropyl)carboxamide, methyl 2-carbonylamino-3-hydroxypropanoate, N-(2-hydroxypropyl)carboxamide, N-(2-hydroxy-isopropyl)carboxamide, N-[2-hydroxy-1-(hydroxymethyl)ethyl]carboxamide, N-(2-carbonylaminoethyl)acetamide, N-(2-(2-pyridyl)ethyl)carboxamide, N-(2-pyridylmethyl)carboxamide, N-(oxolan-2-ylmethyl)-carboxamide, N-(4-hydroxypyrrolidin-2-yl)carboxamide, N-[2-(2-hydroxyethoxy)ethyl]-carboxamide, N-(4-hydroxycyclohexyl)carboxamide, N-[2-(2-oxo-4-imidazolinyl)ethyl]-carboxamide, N-(carbonylaminomethyl)acetamide, N-(3-pyrrolidinylpropyl)carboxamide, N-[1-(carbonylaminomethyl)pyrrolidin-3-yl]acetamide, N-(2-morpholin-4-ylethyl)carboxamide, N-[3-(2-oxopyrrolidinyl)propyl]carboxamide, 4-methyl-2-oxopiperazinecarbaldehyde, N-(2-hydroxy-3-pyrrolidinylpropyl)carboxamide, N-(2-hydroxy-3-morpholin-4-ylpropyl)carboxamide, N-{2-[(5-cyano-2-pyridyl)amino]ethyl}carboxamide, 3-(dimethylamino)pyrrolidinecarbaldehyde, N-[(5-methylpyrazin-2-yl)methyl]carboxamide, 2,2,2-trifluoro-N-(1-formylpyrrolidin-3-yl)acetamide,

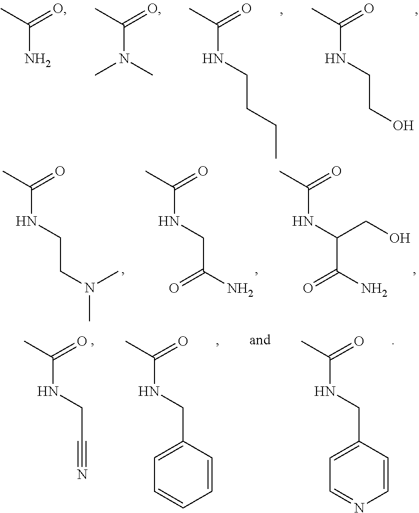

Representative substituted alkoxycarbonyl groups include, for example, those shown below. These alkoxycarbonyl groups can be further substituted as will be apparent to those having skill in the organic and medicinal chemistry arts in conjunction with the disclosure herein.

Representative substituted alkoxycarbonyl groups include, for example, those shown below. These alkoxycarbonyl groups can be further substituted as will be apparent to those having skill in the organic and medicinal chemistry arts in conjunction with the disclosure herein.

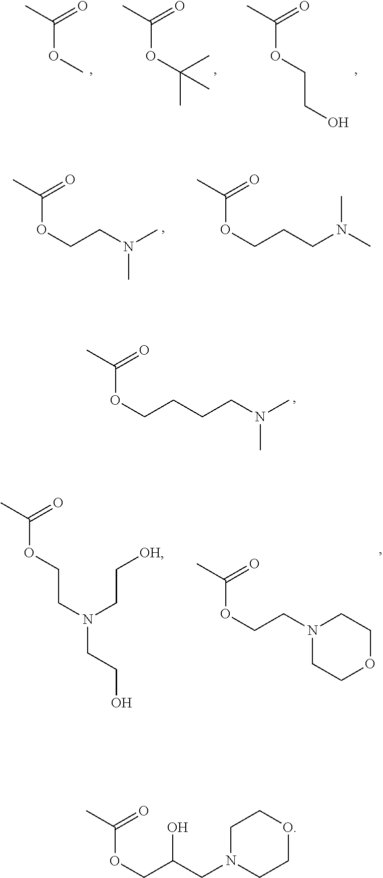

The term “amino” refers herein to the group —NH₂. The term “alkylamino” refers herein to the group —NRR′ where R is alkyl and R′ is hydrogen or alkyl. The term “arylamino” refers herein to the group —NRR′ where R is aryl and R′ is hydrogen, alkyl, or aryl. The term “aralkylamino” refers herein to the group —NRR′ where R is aralkyl and R′ is hydrogen, alkyl, aryl, or aralkyl.

The term “alkoxyalkylamino” refers herein to the group —NR-(alkoxyalkyl), where R is typically hydrogen, aralkyl, or alkyl.

The term “aminocarbonyl” refers herein to the group —C(O)—NH₂. “Substituted aminocarbonyl” refers herein to the group —C(O)—NRR′ where R is alkyl and R′ is hydrogen or alkyl. The term “arylaminocarbonyl” refers herein to the group —C(O)—NRR′ where R is aryl and R′ is hydrogen, alkyl or aryl. “Aralkylaminocarbonyl” refers herein to the group —C(O)—NRR′ where R is aralkyl and R′ is hydrogen, alkyl, aryl, or aralkyl.

The term “amidino” refers to the moieties R—C(═N)—NR′— (the radical being at the “N¹” nitrogen) and R(NR')C═N-(the radical being at the “N²” nitrogen), where R and R′ can be hydrogen, alkyl, aryl, or aralkyl.

As used herein, the term “pharmaceutically acceptable salts” refers to the nontoxic acid or alkaline earth metal salts of the pyrimidine compounds of the invention. These salts can be prepared in situ during the final isolation and purification of the pyrimidine compounds, or by separately reacting the base or acid functions with a suitable organic or inorganic acid or base, respectively. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2-hydroxyethanesulfonate, lactate, maleate, methanesulfonate, nicotinate, 2-naphth-alenesulfonate, oxalate, pamoate, pectinate, persulfate, 3-phenylproionate, picrate, pivalate, propionate, succinate, sulfate, tartrate, thiocyanate, p-toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, hydroboric acid, nitric acid, sulfuric acid and phosphoric acid and such organic acids as formic acid, acetic acid, trifluoroacetic acid, fumaric acid, tartaric acid, oxalic acid, maleic acid, methanesulfonic acid, succinic acid, malic acid, methanesulfonic acid, benzenesulfonic acid, and p-toluenesulfonic acid, citric acid, and acidic amino acids such as aspartic acid and glutamic acid.

Basic addition salts can be prepared in situ during the final isolation and purification of the pyrimidine compounds, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, pyridine, picoline, triethanolamine and the like, and basic amino acids such as arginine, lysine and ornithine.

### IV. Experimental Materials and Methods

Mice:

Rag2^{-/-};Il2rg^{-/-} breeders were kindly given by Drs. T. Nomura and M. Ito of the Central Institute for Experimental Animals (Kawasaki, Japan). Mice were then bred in our animal facilities under sterile conditions. Athymic Crl:CD-1-Foxn1^{nu/nu} mice (referred to as nude mice) were purchased from Charles River Italy and kept under sterile conditions. Experiments were authorized by the institutional review board of the University of Bologna and done according to Italian and European guidelines.

Cell Lines:

MDA-MB-453 and BT-474 breast cancer cell lines were originally obtained from by Dr. Serenella M. Pupa (Istituto Nazionale dei Tumori, Milan, Italy). Cell lines were authenticated by DNA fingerprinting on the 11 Nov. 2010 (performed by DSMZ, Braunschweig, Germany). Cells were routinely cultured in Roswell Park Memorial Institute (RPMI) medium supplemented with 10% foetal bovine serum and were maintained at 37° C. in a humidified 5% CO₂ atmosphere. All medium constituents were purchased from Invitrogen, Milan, Italy. In order to visualize metastatic cells/lesions, breast cancer cell lines were transfected with a plasmid expressing Enhanced Green Fluorescent Protein (pEGFP-N1, Clontech, Mountain View, Calif.) using Lipofectamine 2000 (Invitrogen). Stable transfectants were selected using G418 (Invitrogen). EGFP expression was monitored by fluorescence microscopy and quantified by cytofluorimetric analysis.

Metastasis Induction:

Nine-20-week-old female Rag2^{-/-};Il2rg^{-/-} mice were used throughout this work. Local tumors were induced with 10⁷ viable human tumour cells injected either subcutaneously in the right hind leg or in the fat pad of the fourth (abdominal) left mammary gland in 0.2 ml of PBS. Tumor diameters were periodically measured with digital calipers and tumor volumes calculated as π/2×[√(a×b)]³/6, where a=maximal tumor diameter and b=major tumor diameter perpendicular to a. For intravenous (i.v.) administrations, 2×10⁶ cells in 0.4 ml PBS were injected in a tail vein. Nude mice (5-6 week-old) were treated i.v. with anti-asialo GM1 antiserum (Wako, Dusseldorf, Germany), 0.4 ml of a 1:30 dilution in PBS, to deplete NK activity, 24 h prior to the injection of human tumour cells. Pilot experiments were performed to assess for each cell line the time at which experimental metastases could be detected.

Metastasis Detection and Quantification:

Tumor-bearing mice were sacrificed at various times (see Results), depending on tumor and metastasis growth, and were subjected to an accurate necropsy. Lungs were stained with black India ink to better outline metastases and fixed in Fekete's solution. Lung and liver metastases were counted using a dissection microscope. When EGFP-expressing cells were injected, whole mice and dissected organs were carefully examined using a Lightools imaging system (Lightools Research, Encinitas, Calif.) to detect fluorescent metastatic deposits. Quantification of metastatic load in brain and bone marrow was performed by immunofluorescence followed by cytofluorimetric analysis and by Real-Time PCR. Brain was minced with scissors and passed through a 70 μm cell strainer (Becton Dickinson, Bedford, Mass., USA) to obtain a homogeneous cell suspension. Bone marrow was flushed from both femurs in PBS and filtered through a 70 μm strainer. A mouse monoclonal antibody against human HER-2 (clone Neu 24.7, Becton Dickinson, San Jose, Calif., USA) labeled with phycoerythrin was used to quantify human cells by cytofluorometric analysis. Real-Time PCR was performed on genomic DNA extracted with 10 mM Tris-HCl buffer pH 8.3 containing 50 mM KCl, 2.5 mM MgCl₂, 0.01% gelatin, 0.45% igepal, 0.45% tween 20 and 120 μg/ml proteinase K (all reagents from Sigma, Milan, Italy) by an overnight incubation at 56° C. followed by 30 mM incubation at 95° C. to inactivate the proteinase K. A sequence of the α-satellite region of the human chromosome 17 was amplified. Primer and probe sequences derived from Becker et al. (British Journal of Cancer 2002, 87:1328-1335) with the sole alteration that the probe carried the non-fluorescent quencher dye TAMRA at the 3′-end. 100 ng DNA per sample was amplified using 250 nM primers and 100 nM probe in a final volume of 25 μl of TaqMan Universal PCR Master Mix (Applied Biosystems, Milan, Italy). After an initial denaturation step at 95° C. for 10 mM, 45 cycles of amplification (95° C. 30 sec plus 60° C. 1 min) were performed in a 5700 Sequence Detection System (Applied Biosystems). To quantify human cells, a standard curve was constructed by adding scalar amounts of MDA-MB-453 human cells to cells from the mouse whole brain. Cₜ (threshold cycle) values obtained from the experimental samples were interpolated in the standard curve run in each PCR.

Metastasis Therapy with Compound A:

Compound A was formulated in 1-methyl-2-pyrrolidone (NMP)/poly-ethylene glycol 300 (PEG300) (Fluka) (10/90, v/v). Solutions (7.5 mg/ml) were prepared fresh each day of treatment and carefully shielded from light. Groups of 6-9 Rag2^{-/-};Il2rg^{-/-} female were challenged with 453-EGFP cell i.v. A dose of 50 mg/kg Compound A was given per os daily starting from the day after cell injection, control mice received vehicle alone. Mice received four drug administrations in the first week, and five drug administrations in the following weeks, for a total amount of 37 treatments. Mice were sacrificed 1-4 days after the last treatment.

### V. Experimental Results

Tumor Growth and Metastatic Spread in Rag2^{-/-};Il2rg^{-/-} Mice:

Many interesting human breast cancer cell lines, in particular those expressing HER-2, grow poorly in nude mice, and usually do not metastasize, even if NK activity is temporarily blocked by treatment of the host with NK-depleting antibodies. HER-2⁺ MDA-MB-453 ad BT-474 cells were not tumorigenic in nude mice. The same cells gave rise to progressive local tumors in Rag2^{-/-};Il2rg^{-/-} mice with very short latency times, both after orthotopic (intramammary) and subcutaneous administration.

Metastatic dissemination in Rag2^{-/-};Il2rg^{-/-} mice was widespread, and reached all sites commonly affected in human patients, including lungs, liver, bones and brain. Interestingly, metastatic spread from local tumors was only marginally different between tumors growing orthotopically or subcutaneously, both for what concerned the more malignant MDA-MB-453 cell line and the less malignant BT-474. Intravenous administration of cells significantly enhanced the metastatic spread of BT-474 cell, with the proportion of affected mice attaining 100%.

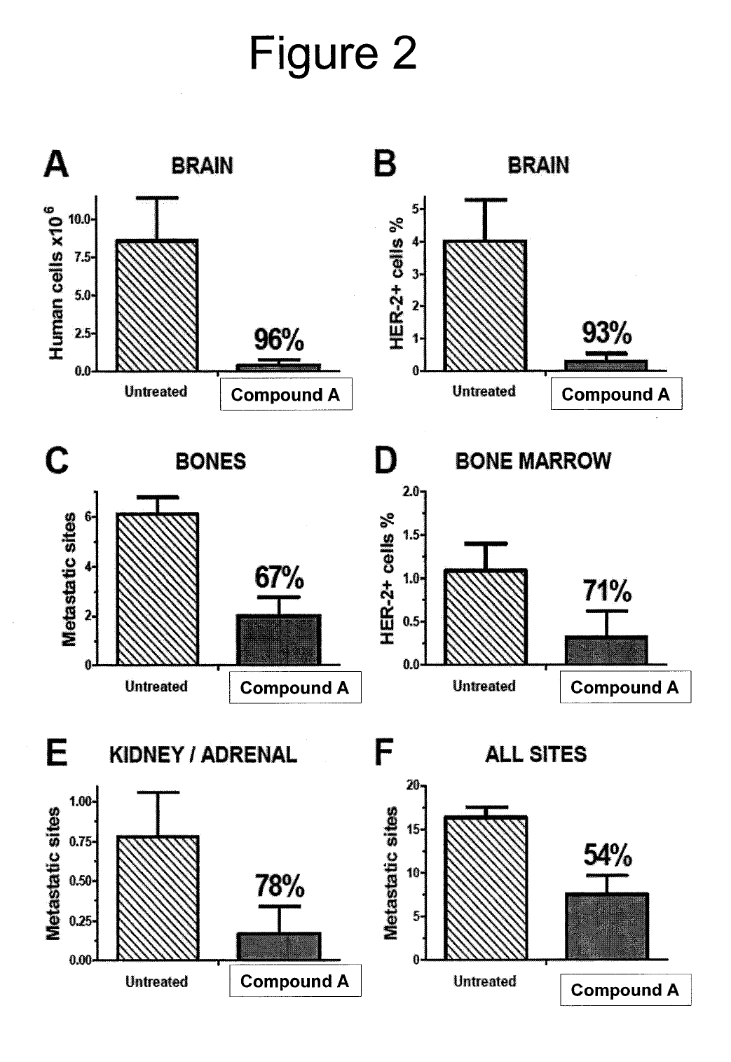

Different metastatic burdens were observed in different organs, and the use of EGFP-tagged cells was instrumental in allowing a precise detection of metastatic deposits in bones, liver and brain. However the evaluation of metastatic spread in Rag2^{-/-};Il2rg^{-/-} mice was not limited to EGFP detection, for example we used qPCR with human-specific primers to quantitate metastatic burden in the brain, and flow cytometry to detect HER-2-positive disseminated tumor cells in the bone marrow (FIG. 2).

Brain Metastases:

The brain is common site of metastatic spread, but tumor growth in immunodeficient mice fails to reproduce this fateful property of human tumors, unless unique cell lines, selected variants and/or special injection routes are used. In contrast, spontaneous brain metastases from local tumors were common in Rag2^{-/-};Il2rg^{-/-} mice, and their frequency reached 100% after intravenous injection. In summary, the Rag2^{-/-};Il2rg^{-/-} mouse is an exquisite model for the study of brain dissemination.

Therapy of Disseminated Human Breast Cancer:

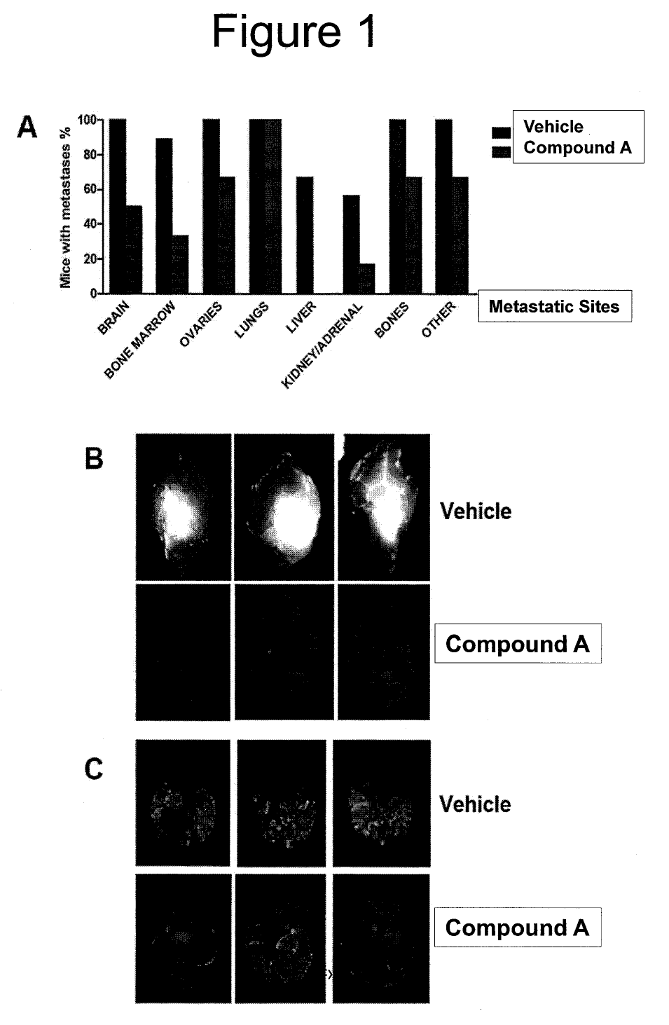

Metastatic dissemination of human breast cancer is a great therapeutic challenge and a moving target, because current therapies have modified the risk of metastatic relapse in different organs, for example monoclonal antibodies seem to enhance the risk of brain metastases (7). Therefore a mouse model of multiorgan metastatic dissemination is an important tool to investigate new antimetastatic drugs. Compound A, a selective PI3K inhibitor with excellent penetration of the blood-brain barrier (8), affected metastatic growth in different target organs of 453-EGFP cells. Compound A produced a highly significant and widespread reduction of the metastatic burden. In multiple sites a sizeable proportion of mice were metastasis-free. (FIG. 1A). The number of bone metastases was decreased by 67% (FIG. 2C). Even in the more heavily colonized organs, such as the brain and lungs, Compound A strongly inhibited the growth of 453-EGFP (FIG. 1B-C). To better assess Compound A therapeutic activity against brain metastases human cells were quantitated using human-specific qPCR or HER-2 flow cytometry. In the brains of mice treated with Compound A, >90% inhibition was found in the number of human cells with both technologies (FIG. 2A-B).

The efficacy of Compound A in controlling metastatic growth in multiple organs, including the brain, forecasts clinical impact in analogous clinical situations.

### VI. References

1. Nguyen D X, Bos P D, Massague J. Metastasis: from dissemination to organ-specific colonization. Nat Rev Cancer. 2009; 9(4):274-84.
2. Langley R R, Fidler I J. The seed and soil hypothesis revisited—The role of tumor-stroma interactions in metastasis to different organs. Int J. Cancer. 2011; 128(11):2527-35.
3. Jonkers J, Derksen P W. Modeling metastatic breast cancer in mice. J Mammary Gland Biol Neoplasia. 2007; 12(2-3):191-203.
4. Weigelt B, Peterse J L, van't Veer L J. Breast cancer metastasis: markers and models. Nat Rev Cancer. 2005; 5(8):591-602.
5. Weilbaecher K N, Guise T A, McCauley L K. Cancer to bone: a fatal attraction. Nat Rev Cancer. 2011; 11(6):411-25.
6. Lee Y T. Breast carcinoma: pattern of metastasis at autopsy. J Surg Oncol. 1983; 23(3): 175-80.
7. Steeg P S, Camphausen K A, Smith Q R. Brain metastases as preventive and therapeutic targets. Nat Rev Cancer. 2011; 11(5):352-63.
8. Sanchez C G, Ma C X, Crowder R J, et al. Preclinical modeling of combined phosphatidylinositol-3-kinase inhibition with endocrine therapy for estrogen receptor-positive breast cancer. Breast Cancer Res. 2011; 13 (2):R21.

1. A method for the treatment of bone cancer in a subject in need of such treatment, comprising administering a compound according to formula (I): 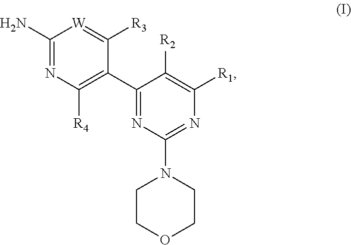
wherein
W represents CH;
R¹ represents N-morpholinyl;
R² represents hydrogen;
R³ represents trifluoromethyl;
R⁴ represents hydrogen,
or a stereoisomer, tautomer, or pharmaceutically acceptable salt thereof, to the subject such that the bone cancer is treated.

2. (canceled)

3. The method of claim 1, wherein the bone cancer is selected from chondrosarcoma, osteosarcoma, Ewing's sarcoma, chordoma, fibrosarcoma, and malignant fibrous histiocytoma (MFH).

4. A method of preventing the metastatic dissemination of primary cancer cells into the bone of a subject in need of such prevention, comprising administering a compound according to formula (I) according to claim 1, or a stereoisomer, tautomer, or pharmaceutically acceptable salt thereof, to the subject such that the metastatic dissemination of primary cancer cells into the bone of the subject is prevented.

5. (canceled)

6. The method of claim 4, wherein the primary cancer cells originate from cancers of the breast, lung, pancreas, kidney or prostate.

7. The method of claim 4, wherein the primary cancer cells are breast cancer cells.

8. A method of claim 1 or 4, wherein the compound according to formula (I) is administered with a pharmaceutically acceptable carrier, diluent or excipient, to the subject thereof.

9. (canceled)

10. (canceled)

11. The method according to claim 1, wherein the bone cancer is metastatic bone cancer.

12. (canceled)
